(19) United States
(12) Patent Application Publication
(10) Pub. No.: US 2023/0086047 A1
(43) Pub. Date: Mar. 23, 2023

(54) PLATFORM FOR MAPPING COMPOUNDS FOR PRE-CLINICAL EFFECTIVENESS TO PREDICT CLINICAL EFFECTIVENESS FOR TREATING CONDITIONS

(71) Applicant: Verséa Holdings Inc., Tampa, FL (US)

(72) Inventors: Stephen Porada, Kinnelon, NJ (US); Toby Moeller-Bertram, Tampa, FL (US); Sean Fetcho, Tampa, FL (US)

(21) Appl. No.: 17/948,834

(22) Filed: Sep. 20, 2022

Related U.S. Application Data
(60) Provisional application No. 63/246,185, filed on Sep. 20, 2021.

(51) Int. Cl. G16H 10/20; G16H 70/40

(52) U.S. Cl. CPC G16H 10/20; G16H 70/40

(57) ABSTRACT

Provided herein is a discovery platform, methods, computer program products, and systems for translating and predicting pre-clinical results of pharmaceutical compounds. The platform allows, for example, evaluation of existing compounds to identify quality candidate compounds to move to pre-clinical studies.

### CROSS-REFERENCE TO RELATED APPLICATIONS

The present application claims priority to U.S. Provisional Application No. 63/246,185 filed Sep. 20, 2021, the full disclosure of which is incorporated by reference in its entirety for all purposes.

### BACKGROUND

Drug development is a time-consuming and costly process. Candidate drugs must be evaluated both for safety and effectiveness through extensive and expensive clinical trials. Before reaching the clinical trial stage, drug developers have traditionally evaluated a large number of compounds in mechanistic preclinical studies to select candidate compounds. Even for drugs that make it to market, the process is costly, and the multitude of candidate compounds that fail to make it through clinical trials (i.e., up to 85% of candidate compounds that begin clinical trials) only add to the overall cost.

U.S. Pre-Grant Publication No. 2021/0057050 describes a computer-implemented method can include: receiving input of a biological target; receiving a generative model (e.g., tensorial reinforcement learning (GENTRL) model or other model) trained with reference compounds, wherein the reference compounds include: general compounds, compounds that modulate the biological target, and compounds that modulate biomolecules other than the biological target; generating structures of generated compounds with the generative model; prioritizing structures of generated compounds based on at least one criteria; processing prioritized chemical structures of the generated compounds through a Sammon mapping protocol to obtain hit structures; and providing chemical structures of the hit structures. One or more non-transitory computer readable media are provided that store instructions that in response to being executed by one or more processors, cause a computer system to perform operations, the operations comprising performing the computer methods described in the method for providing chemical structure of hit structures generated by the generative model.

Paul et al., in Artificial intelligence in drug discovery and development, Drug Discovery Today, 2021, 26(1) 80-93, published Oct. 21, 2020, explain that the use of artificial intelligence (AI) has been increasing in various sectors of society, particularly the pharmaceutical industry. In the review, they highlight the use of AI in diverse sectors of the pharmaceutical industry, including drug discovery and development, drug repurposing, improving pharmaceutical productivity, and clinical trials, among others; such use reduces the human workload as well as achieving targets in a short period of time. They also discuss crosstalk between the tools and techniques utilized in AI, ongoing challenges, and ways to overcome them, along with the future of AI in the pharmaceutical industry.

Yang et al., in Concepts of Artificial Intelligence for Computer-Assisted Drug Discovery, Chem. Rev. 2019, 119, 18, 10520-10594, published on Jul. 11, 2019, explains that Artificial intelligence (AI), and, in particular, deep learning as a subcategory of AI, provides opportunities for the discovery and development of innovative drugs. Various machine learning approaches have recently (re)emerged, some of which may be considered instances of domain-specific AI which have been successfully employed for drug discovery and design. This review provides a comprehensive portrayal of these machine learning techniques and of their applications in medicinal chemistry. After introducing the basic principles, alongside some application notes, of the various machine learning algorithms, the current state-of-the art of AI-assisted pharmaceutical discovery is discussed, including applications in structure- and ligand-based virtual screening, de novo drug design, physicochemical and pharmacokinetic property prediction, drug repurposing, and related aspects. Finally, several challenges and limitations of the current methods are summarized, with a view to potential future directions for AI-assisted drug discovery and design.

Vamathevan et al., in Applications of machine learning in drug discovery and development, Nature Reviews Drug Discovery, 17, 463-477 (2019), published Apr. 11, 2019, explains that drug discovery and development pipelines are long, complex and depend on numerous factors. Machine learning (ML) approaches provide a set of tools that can improve discovery and decision making for well-specified questions with abundant, high-quality data. Opportunities to apply ML occur in all stages of drug discovery. Examples include target validation, identification of prognostic biomarkers and analysis of digital pathology data in clinical trials. Applications have ranged in context and methodology, with some approaches yielding accurate predictions and insights. The challenges of applying ML lie primarily with the lack of interpretability and repeatability of ML-generated results, which may limit their application. In all areas, systematic and comprehensive high-dimensional data still need to be generated. With ongoing efforts to tackle these issues, as well as increasing awareness of the factors needed to validate ML approaches, the application of ML can promote data-driven decision making and has the potential to speed up the process and reduce failure rates in drug discovery and development.

Despite the above disclosures, there is a need for methods and systems to evaluate existing compounds to identify quality candidate compounds and predict pre-clinical and clinical results, reducing the failure rate and the overall cost of drug development.

### BRIEF SUMMARY

Provided herein are methods for predicting a cellular response to a pharmaceutical compound or a plurality of pharmaceutical compounds. In some embodiments, the methods comprise obtaining a set of functional assay data for the compound or plurality of pharmaceutical compounds. In some embodiments, the functional assay data are from one or more functional assays performed on cells in cell culture. In some embodiments, the one or more functional assays comprise a cell viability assay, a cytokine production assay, a mitochondrial function assay, a reactive oxygen/nitrogen species generation assay, a cyclic-AMP synthesis assay, a gene expression assay, a cell immune response assay, or any combination thereof. In some embodiments, the methods provided herein further comprise predicting a therapeutic outcome for the pharmaceutical compound or plurality of pharmaceutical compounds. In some embodiments, the pharmaceutical compound or plurality of pharmaceutical compounds comprises at least one existing medication.

Also provided herein are computer-program products and systems for predicting a cellular response to a pharmaceutical compound or a plurality of pharmaceutical compounds.

Also provided herein are methods for identifying significant correlations between physiochemical features of a plurality of pharmaceutical compounds and cellular responses to those compounds. In some embodiments, the methods comprise obtaining a set of functional assay data for a plurality of pharmaceutical compounds. In some embodiments, the functional assay data are based on one or more functional assays performed on each of a plurality of populations of cells. In some embodiments, each of the plurality of populations of cells has independently been contacted with a dose of one of the plurality of pharmaceutical compounds. In some embodiments, the methods provided herein further comprise calculating, for each of the one or more functional assays, a parameter describing a correlation between (1) one or more physiochemical features of the plurality of pharmaceutical compounds, and (2) the functional assay data for the functional assay. In some embodiments, the parameter comprises a P-value. In some embodiments, the one or more physiochemical features comprise the molecular weight of each of the plurality of pharmaceutical compounds, the polar surface area of each of the plurality of pharmaceutical compounds, the hydrogen bond donor count of each of the plurality of pharmaceutical compounds, the hydrogen bond acceptor count of each of the plurality of pharmaceutical compounds, the complexity score of each of the plurality of pharmaceutical compounds, the rotatable bond count of each of the plurality of pharmaceutical compounds, the octanol-water partition coefficient of each of the plurality of pharmaceutical compounds, the aqueous solubility of each of the plurality of pharmaceutical compounds, the pKa value of each of the plurality of pharmaceutical compounds, the plasma protein binding score of each of the plurality of pharmaceutical compounds, or a combination thereof. In some embodiments, the methods provided herein further include determining which of the calculated parameters satisfy a significance threshold.

Also provided herein are computer-program products and systems for identifying significant correlations between physiochemical features of a plurality of pharmaceutical compounds and cellular responses to those compounds. The computer products and systems comprise a non-transitory machine-readable storage medium, including instructions configured to cause one or more data processors to perform actions including the steps of any of the methods disclosed herein for identifying significant correlations between physiochemical features of a plurality of pharmaceutical compounds and cellular responses to those compounds.

### BRIEF DESCRIPTION OF THE DRAWINGS

The present application includes the following figures. The figures are intended to illustrate certain embodiments and/or features of the methods and systems, and to supplement any description(s) of the methods and systems. The figures do not limit the scope of the methods and systems, unless the written description expressly indicates that such is the case.

FIG. 1 is a schematic illustration of multi-mode in vitro testing leading to candidate selection and in vivo testing, according to aspects of this disclosure.

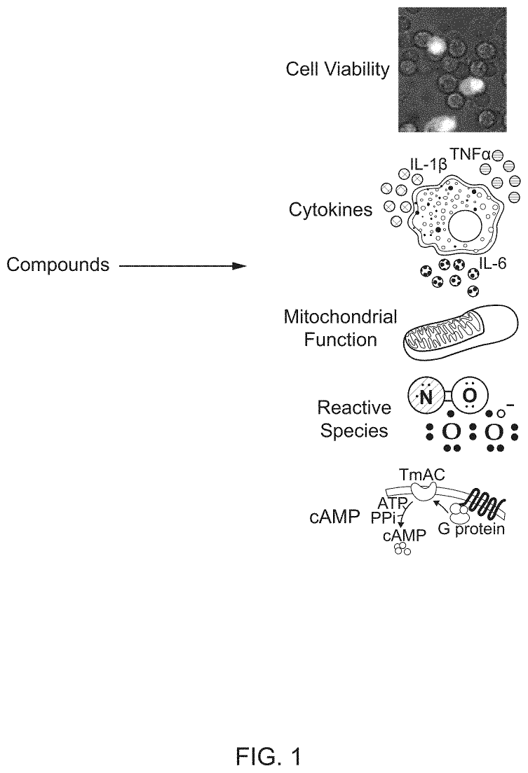

FIG. 2 is a graph plotting the ranges of molecular weights, polar surface areas, hydrogen bond donor counts, and complexity scores for twelve pharmaceutical compounds selected according to certain aspects of this disclosure.

FIG. 3 is a series of graphs plotting functional assay data based on oxidative phosphorylation, glycolysis, mitochondrial respiration, and mitochondrial ATP synthesis assays performed on cell populations contacted with one of six different doses of the twelve selected pharmaceutical compounds of FIG. 2 according to certain aspects of this disclosure.

FIG. 4 is a series of heat maps visualizing the functional assay data of FIG. 3.

FIG. 5 is a graph plotting a correlation between the glycolysis assay data of FIG. 3 and selected physiochemical features, including molecular weights, of the twelve selected compounds of FIG. 2. The shading of the points plotted in the graph corresponds to the molecular weights of the compounds, also indicated on the x-axis of the graph. The diameters of the points correspond to the hydrogen bond acceptor counts, ranging from 2 to 22, of the compounds.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that dual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

### I. Introduction

Provided herein is a discovery platform for translating and predicting pre-clinical results of pharmaceutical compounds. The platform allows evaluation of existing compounds to identify quality candidate compounds to move to pre-clinical studies. By allowing selection of quality candidate compounds, the platform reduces the failure rate and the overall cost of drug development. As described herein, the platform comprises methods, computer program products, and systems comprising machine learning algorithms to predict the effect of pharmaceutical compounds on cell-based pathways that are common to diseases, leveraging an in vitro, cell culture-based human body model as a combination of high-throughput, multi-endpoint functional in vitro assays that model cellular responses in multiple clinical conditions. The platform may be applied to broad pharmacological discovery, irrespective of the particular compounds being tested and particular disease processes. In addition, the platform allows personalization of compound effect prediction by development of disease process models and application of test compounds to normal models and comparison to application of test compounds to diseased models (e.g., cancer models, heart disease models, neurodegeneration models, etc.).

An example application of the platform is the screening of multiple biologics and pharmaceutical compounds to assist in the identification of the most viable candidates to further develop based on therapeutic signal and safety characterization and quantification. The compound may then be compared to known compounds, to judge toxicity, efficacy, etc. The platform may further be used to predict side effects/interactions of the candidate with existing medications.

### II. Cellular Response Prediction Methods

In one aspect, provided herein are methods for predicting a cellular response to a pharmaceutical compound or a plurality of pharmaceutical compounds. In some embodiments, the methods are computer implemented methods. As used herein, a “pharmaceutical compound” is any substance (other than food) intended to affect any structure or any function of the body of a subject (e.g., a human subject or a non-human subject). Pharmaceutical compounds may be a small molecule (e.g., a chemically synthesized small molecule drug), a biologic product (e.g., a recombinant protein, a vaccine, a blood product, a gene therapy product, or a stem cell therapy product), a natural product (e.g., a chemical compound or substance produced by a living organism), isolated components of any of the above, biosimilars of any of the above, or combinations of any of the above. In some embodiments, the pharmaceutical compound is an existing compound (e.g., an FDA-approved drug, a compound in a drug-screening library, a compound in a small molecule library, a commercially available small compound, a biological product, etc.). Pharmaceutical compounds may be formulated for administration to a subject via oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump.

In some embodiments, the plurality of pharmaceutical compounds comprises existing compounds. In some embodiments, the plurality of pharmaceutical compounds comprises at least one non-naturally-occurring compound. In some embodiments, the plurality of pharmaceutical compounds consists of a plurality of non-naturally-occurring compounds. A pharmaceutical compound of the present disclosure may include any pharmacological agent that may be solubilized in aqueous media as well as various other suitable solvents (e.g., ethanol, DMSO), genetic material (e.g., viral vectors, miRNA, antisense), or any biological material (e.g., plasma, serum, isolated factors, exosomes).

It is also understood that the methods and systems disclosed herein are equally applicable for predicting a cellular response to a living cell, tissue, or organism or to a plurality of living cells, tissues or organisms. The cells or tissues can be human or non-human. The cells or tissues can be autologous, homologous, or heterologous. For example, the provided methods and systems may be used to examine responses to one or more living cells, tissues, or organisms for identifying one or more correlations between the responses and the living cells, tissues, or organisms. The provided methods and systems may additionally or alternatively be used for generating and applying predictive models based on those correlations. Accordingly, whenever the methods or systems described herein refer to a pharmaceutical compound or a plurality of pharmaceutical compounds, this reference is intended to also encompass living cells, tissues, or organisms.

In some embodiments, the methods comprise obtaining a set of functional assay data for a pharmaceutical compound or a plurality of pharmaceutical compounds. In some embodiments, the functional assay data are results based on performance of one or more functional assays. In some embodiments, the one or more functional assays are performed on cells in culture. In some embodiments, the cells in culture are a plurality of populations of cells, and each of the plurality of populations of cells has independently been contacted with a dose of one of the plurality of pharmaceutical compounds. In some embodiments, the one or more functional assays include a cell viability assay, a cytokine production assay, a mitochondrial function assay, a reactive oxygen/nitrogen species generation assay, a cyclic-AMP synthesis assay, a gene expression assay, a cell immune response assay, or any combination thereof. In some embodiments, other functional assays may be useful.

In some embodiments, the set of functional assay data include performance results of one or more functional assays, where different populations of cells are each independently exposed to a dose of a different pharmaceutical compound of the plurality of pharmaceutical compounds, and where at least one population of cells is not exposed to a pharmaceutical compound and serves as a control. In some embodiments, the functional assay data includes performance results measuring the differences between a control population response and the responses for each of the populations of cells contacted with one of the plurality of pharmaceutical compounds.

In some embodiments, the methods provided herein comprise selecting the dose of the plurality of pharmaceutical compounds such that the various differences between a control population response and the non-control responses define a sufficiently broad absolute range, where the absolute range is the difference between the highest and lowest percent differences among the percent differences from control for the non-control responses. By selecting the dose according to this criterion, correlations between the obtained functional assay data and one or more physiochemical features of the compounds may be improved. In particular, a calculated correlation may be less subject to experimental noise, may have greater statistical significance, and may be predictive of a cellular response to a wider variety of additional existing or potential pharmaceutical compounds. The dose may be selected such that the absolute range of the percent differences from control for the functional assay data is, for example, greater than 10%, e.g., greater than 15%, greater than 20%, greater than 25%, greater than 30%, greater than 40%, greater than 50%, greater than 55%, or greater than 60%. Smaller ranges, e.g., less than 10%, are also contemplated.

In some embodiments, the methods comprise obtaining a set of functional assay data for at least two of (e.g., at least three of, at least four of, at least five of, at least six of, or at least seven of) a cell viability assay, a cytokine production assay, a mitochondrial function assay, a reactive oxygen/nitrogen species generation assay, a cyclic-AMP synthesis assay, a gene expression assay, a cell immune response assay, or any combination thereof. Any individual combination of functional assays may be selected (e.g., according to the optimization methods described herein below). In some embodiments, the methods comprise obtaining a set of functional assay data for a cell viability assay, a cytokine production assay, a mitochondrial function assay, a reactive oxygen/nitrogen species generation assay, a cyclic-AMP synthesis assay, a gene expression assay, and a cell immune response assay.

In some embodiments, the methods comprise obtaining a set of functional assay data for at least two of (e.g., at least three of, at least four of, or at least give of) a cell viability assay, a cytokine production assay, a mitochondrial function assay, a reactive oxygen/nitrogen species generation assay, a cyclic-AMP synthesis assay, or any combination thereof. Any individual combination of functional assays may be selected (e.g., according to the optimization methods described herein below). In some embodiments, the methods comprise obtaining a set of functional assay data for a cell viability assay, a cytokine production assay, a mitochondrial function assay, a reactive oxygen/nitrogen species generation assay, and a cyclic-AMP synthesis assay.

In some embodiments, the methods comprise obtaining a set of functional assay data for one or more functional assays. In some embodiments, the one or more functional assays are selected from (A) a cell viability assay, (B) a cytokine production assay, (C) a mitochondrial function assay, (D) a reactive oxygen/nitrogen species generation assay, (E) a cyclic-AMP synthesis assay, (F) a gene expression assay, or (G) a cell immune response assay. In some embodiments, the one or more functional assays are A and B; A and C; A and D; A and E; A and F; A and G; B and C; B and D; B and E; B and F; B and G; C and D; C and E; C and F; C and G; D and E; D and F; D and G; E and F; E and G; F and G; or any combination thereof.

In some embodiments, the one or more functional assays are A, B, and C; A, B, and D; A, B, and E; A, B, and F; A, B, and G; A, C, and D; A, C, and E; A, C, and F; A, C, and G; A, D, and E; A, D, and F; A, D, and G; A, E, and F; A, E, and G; A, F, and G; B, C, and D; B, C, and E; B, C, and F; B, C, and G; B, D, and E; B, D, and F; B, D, and G; B, E, and F; B, E, and G; B, F, and G; C, D, and E; C, D, and F; C, D, and G; C, E, and F; C, E, and G; C, F, and G; D, E, and F; D, E, and G; D, F, and G; E, F, and G; or any combination thereof.

In some embodiments, the one or more functional assays are A, B, C, and D; A, B, C, and E; A, B, C, and F; A, B, C, and G; A, B, D, and E; A, B, D, and F; A, B, D, and G; A, B, E, and F; A, B, E, and G; A, B, F, and G; A, C, D, and E; A, C, D, and F; A, C, D, and G; A, C, E, and F; A, C, E, and G; A, C, F, and G; A, D, E, and F; A, D, E, and G; A, D, F, and G; A, E, F, and G; B, C, D, and E; B, C, D, and F; B, C, D, and G; B, C, E, and F; B, C, E, and G; B, C, F, and G; B, D, E, and F; B, D, E, and G; B, D, F, and G; B, E, F, and G; C, D, E, and F; C, D, E, and G; C, D, F, and G; C, E, F, and G; D, E, F, and G; or any combination thereof.

In some embodiments, the one or more functional assays are A, B, C, D, and E; A, B, C, D, and F; A, B, C, D, and G; A, B, C, E, and F; A, B, C, E, and G; A, B, C, F, and G; A, B, D, E, and F; A, B, D, E, and G; A, B, D, F, and G; A, B, E, F, and G; A, C, D, E, and F; A, C, D, E, and G; A, C, D, F, and G; A, C, E, F, and G; A, D, E, F, and G; B, C, D, E, and F; B, C, D, E, and G; B, C, D, F, and G; B, C, E, F, and G; B, D, E, F, and G; C, D, E, F, and G; or any combination thereof.

In some embodiments, the one or more functional assays are A, B, C, D, E, and F; A, B, C, D, E, and G; A, B, C, D, F, and G; A, B, C, E, F, and G; A, B, D, E, F, and G; A, C, D, E, F, and G; B, C, D, E, F, and G; or any combination thereof.

In some embodiments, the one or more functional assays are A, B, C, D, E, F, and G.

In some embodiments, the methods provided herein comprise obtaining cell viability assay data. A cell viability assay estimates the number of viable cells in a population of cells (e.g., a population of cells in a well or multiple wells of a multi-well cell culture plate). Such assays may be used, for example, to assess the effect of a pharmaceutical compound or a plurality of pharmaceutical compounds on cell proliferation or cytotoxicity. Many cell viability assays useful in the methods disclosed herein are known in the art, including, but not limited to, tetrazolium reduction assays, resazurin reduction assays, protease viability marker assays, ATP assays, sodium-potassium ratio assays, cytolysis or membrane leakage assays, mitochondrial activity or caspase activity assays, functional assays, genomic and proteomic assays, flow cytometry assays, lactate dehydrogenase release assays, annexin V/propidium iodide fluorescence assays, or any combination thereof. In some embodiments, the cell viability assay data obtained in the methods provided herein are from a colorimetric MTT assay. MTT (3-(4,5-dimethylthiazol-2-yl)-2,5-diphenyltetrazolium bromide) is a yellow tetrazole that is reduced to purple formazan in living cells, allowing colorimetric readout of cell viability in cells treated with MTT. Suitable MTT assay kits are commercially available (e.g., the MTT assay kit from Abcam, catalog number ab211091).

In some embodiments, the methods provided herein comprise obtaining cytokine production assay data. A cytokine production assay involves quantitative and/or qualitative assessment of cytokine production and/or secretion from a population of cells. In the methods provided herein, production may be assessed for any cytokine of interest, including, but not limited to, IL-1β, TNFα, IL-6, or combinations thereof. In some embodiments, multiplex assays are used to detect anywhere from 2 to several hundred or more cytokines (e.g., Multiplex Cytokine Assay Kits available from R&D Systems, Inc.). Cytokine production assays suitable for the methods disclosed herein are known in the art, including, but not limited to, immunoassays (e.g., western blots, dot blots), bioassays, protein microarray assays, liquid chromatography assays, sandwich enzyme-linked immunosorbent assays (ELISAs), mRNA quantification assays (e.g., real-time PCR, RNA-seq), electrochemiluminescence assays, bead-based multiplex immunoassays (MIA), or any combination thereof. In some embodiments, the cytokine production assay data obtained in the methods provided herein are from a cytokine-specific ELISA. In some embodiments, the data are from real-time PCR analysis of cytokine mRNA. In some embodiments, the cytokines measured comprise IL-1β, TNFα, IL-6, or combinations thereof.

In some embodiments, the methods provided herein comprise obtaining mitochondrial function assay data. Mitochondrial function assays generally involve quantitative and/or qualitative assessment of at least one aspect of mitochondrial function (e.g., membrane potential, superoxide production, calcium levels, permeability, etc.). Suitable mitochondrial function assays are known in the art and include, but are not limited to, fluorescence assays (e.g., to detect mitochondrial calcium, superoxide, mitochondrial permeability transition, membrane potential, etc.), metabolic fuel flux assays, oxygen consumption assays, ATP assays, glycolysis assays, or any combination thereof. These and other suitable mitochondrial function assays are described in Connolly et al., 2017, Cell Death Differ. 25(3):542-572. In some embodiments, the mitochondrial function assay data obtained in the methods provided herein are from analysis of mitochondrial respiration via metabolic flux analysis. In some embodiments, the analysis is performed using a commercially available instrument, e.g., the Seahorse XFe96 Metabolic Flux analyzer available from Agilent.

In some embodiments, the methods provided herein comprise obtaining reactive oxygen/nitrogen species generation assay data. Reactive oxygen species (ROS) and reactive nitrogen species (RNS) are relatively unstable oxygen- or nitrogen-centered free radicals that contain unpaired electrons, and they are generated endogenously in cells during the course of normal metabolism, during disease development, and in response to tissue injury or stress. Suitable assays for quantifying ROS and RNS are known in the art and include, but are not limited to, chemical assays, chemiluminescent assays, fluorescence assays, direct or spin trapping-based electron paramagnetic resonance (EPR) spectroscopy, or any combination thereof. In some embodiments, the ROS and RNS generation assay data obtained in the methods provided herein are from EPR assays and/or live-cell plate reader assays. In some embodiments, the live-cell plate reader assays comprise measurement of mitochondrial superoxide and/or nitric oxide. In some embodiments, live-cell plate reader assays are performed using commercially available kits (e.g., the MitoSOX assay from Thermo Fisher or the OxiSelect NO assay from Cell Biolabs).

In some embodiments, the methods provided herein comprise obtaining cyclic AMP synthesis assay data. Cyclic AMP is a classic second messenger system utilized by cells to initiate signaling cascades. Suitable assays for quantifying cyclic AMP are known in the art and include, but are not limited to, ELISAs, immunoassays, radioimmunoassays, colorimetric assays, fluorescence assays, and any combination thereof. In some embodiments, the cyclic AMP synthesis assay data obtained in the methods provided herein are from radioimmunoassays.

In some embodiments, the methods provided herein comprise obtaining gene expression assay data. Expression may be quantified for any gene of interest. In some embodiments, expression levels for all genes (or all expressed genes) are quantified. Gene expression quantification methods are known in the art, and include, but are not limited to, RNA sequencing, whole genome arrays, real-time PCR, protein assays (e.g., western blot, mass spectroscopy, proteomics, etc.), and microscopic assays (e.g., RNA fluorescence in situ hybridization).

In some embodiments, the methods provided herein comprise obtaining cell immune response assay data. Cell immune response assays generally involve quantitative and/or qualitative assessment of the immune response of a population of cells (e.g., cells in a well or multiple wells of a multi-well cell culture plate) to immunological stimulation. Various cell immune response assays are known in the art, including, but not limited to, T cell-dependent antibody response assays (e.g., via ELISA), flow cytometry assays (e.g., to measure T cell activation or T cell populations), complement assays, cell proliferation assays, natural killer cell activity assays, ELISpot assays, FluoroSpot assays, immunohistochemistry assays, immunoassays, and any combination thereof.

In some embodiments, the methods provided herein comprise calculating, for each of the one or more functional assays, one or more parameters describing a correlation between one or more physiochemical features of the plurality of pharmaceutical compounds, and the functional assay data for the functional assay. For example, the one or more parameters may include a statistical P-value calculated for the correlation. The methods may further include determining which of the calculated parameters satisfy a significance threshold. For example, the determining may include evaluating which of the calculated P-values are less than 0.05. e.g., less than 0.04, less than 0.03, less than 0.02, less than 0.01, less than 0.08, less than 0.06, less than 0.04, less than 0.02, or less than 0.01. In this way, the methods may advantageously be used to identifying significant correlations between physiochemical features of a plurality of pharmaceutical compounds and cellular responses to those compounds. These identified significant correlations may be used to gain insight into relationships between structural and chemical properties of compounds, and the cellular responses that the compounds elicit. The correlations may also be used to predict the cellular responses that would result from contacting cells with one or more other test compounds having known or readily quantifiable physiochemical features.

In some embodiments, the populations of cells contacted with plurality of pharmaceutical compounds include cell populations derived from one or more subjects having known ages, e.g., ages less than 10 years of age, ages between 10 and 20 years of age, ages between 20 and 30 years of age, ages between 30 and 40 years of age, ages between 40 and 50 years of age, ages between 50 and 60 years of age, ages between 60 and 70 years of age, ages between 70 and 80 years of age, and/or ages greater than 80 years of age. In this way, significant correlations identified between structural and chemical properties of compounds, and the cellular responses that the compounds elicit, may be stratified or categorized to describe one or more correlations representative of the age of a subject. In some embodiments, the populations of cells contacted with plurality of pharmaceutical compounds include cell populations derived from one or more subjects having known healthy or diseased status with respect to one or more ailments or disorders. In this way, significant correlations identified between structural and chemical properties of compounds, and the cellular responses that the compounds elicit, may be stratified or categorized to describe one or more correlations representative of the healthy or diseased status of a subject. In some embodiments, the one or more identified correlations describe a baseline normal cellular response stratified by age or underlying disease state.

In some embodiments, the one or more physiochemical features of the plurality of pharmaceutical compounds include the molecular weight of each of the plurality of pharmaceutical compounds. The molecular weight of a compound is the sum of all atomic weights of the constituent atoms in the compound. The molecular weight may be measured in terms of g/mol.

In some embodiments, the methods provided herein comprise selecting the plurality of pharmaceutical compounds such that the different molecular weights of the selected compounds define a sufficiently broad range, where the range is the difference between the highest and lowest molecular weights among the molecular weights of the selected compounds. By selecting the pharmaceutical compounds according to this criterion, the correlation between the obtained functional assay data and one or more physiochemical compound features including molecular weights may be improved. In particular, a calculated correlation may be less subject to experimental noise, may have greater statistical significance, and may be predictive of a cellular response to a wider variety of additional existing or potential pharmaceutical compounds. The plurality of pharmaceutical compounds may be selected such that the range of the molecular weights of the plurality of pharmaceutical compounds is, for example, greater than 500 g/mol, e.g., greater than 625 g/mol, greater than 750 g/mol, greater than 875 g/mol, greater than 1000 g/mol, greater than 1125 g/mol, greater than 1250 g/mol, greater than 1375 g/mol, or greater than 1500 g/mol. Smaller ranges, e.g., less than 500 g/mol, are also contemplated.

In some embodiments, the one or more physiochemical features of the plurality of pharmaceutical compounds include the polar surface area of each of the plurality of pharmaceutical compounds. The polar surface area of a compound corresponds to nitrogen and oxygen atoms of the compound and may be calculated from 2D structure information using contributions from pre-defined structure fragments containing these atoms. The polar surface area may be measured in terms of square angstroms per molecule.

In some embodiments, the methods provided herein comprise selecting the plurality of pharmaceutical compounds such that the different polar surface areas of the selected compounds define a sufficiently broad range, where the range is the difference between the highest and lowest polar surface areas among the polar surface areas of the selected compounds. By selecting the pharmaceutical compounds according to this criterion, the correlation between the obtained functional assay data and one or more physiochemical compound features including polar surface areas may be improved. In particular, a calculated correlation may be less subject to experimental noise, may have greater statistical significance, and may be predictive of a cellular response to a wider variety of additional existing or potential pharmaceutical compounds. The plurality of pharmaceutical compounds may be selected such that the range of the polar surface areas of the plurality of pharmaceutical compounds is, for example, greater than 200 square angstroms per molecule, e.g., greater than 250 square angstroms per molecule, greater than 300 square angstroms per molecule, greater than 350 square angstroms per molecule, greater than 400 square angstroms per molecule, greater than 450 square angstroms per molecule, greater than 500 square angstroms per molecule, greater than 550 square angstroms per molecule, or greater than 600 square angstroms per molecule. Smaller ranges, e.g., less than 200 square angstroms per molecule, are also contemplated.

In some embodiments, the one or more physiochemical features of the plurality of pharmaceutical compounds include the hydrogen bond donor count of each of the plurality of pharmaceutical compounds. The hydrogen bond donor count of a compound may be the sum of nitrogen and oxygen atoms of the compound with at least one implicit or explicit directly bonded hydrogen atom.

In some embodiments, the methods provided herein comprise selecting the plurality of pharmaceutical compounds such that the different hydrogen bond donor counts of the selected compounds define a sufficiently broad range, where the range is the difference between the highest and lowest hydrogen donor bond counts among the hydrogen donor bond counts of the selected compounds. By selecting the pharmaceutical compounds according to this criterion, the correlation between the obtained functional assay data and one or more physiochemical compound features including hydrogen donor bond counts may be improved. In particular, a calculated correlation may be less subject to experimental noise, may have greater statistical significance, and may be predictive of a cellular response to a wider variety of additional existing or potential pharmaceutical compounds. The plurality of pharmaceutical compounds may be selected such that the range of the hydrogen bond donor counts of the plurality of pharmaceutical compounds is, for example, greater than 5, e.g., greater than 7, greater than 9, greater than 11, greater than 13, greater than 15, greater than 17, greater than 19, or greater than 21. Smaller ranges, e.g., less than 5, are also contemplated.

In some embodiments, the one or more physiochemical features of the plurality of pharmaceutical compounds include the hydrogen bond acceptor count of each of the plurality of pharmaceutical compounds. The hydrogen acceptor donor count of a compound may be the sum of all oxygen atoms of the compound and the nitrogen atoms of the compound without any implicit or explicit directly bonded hydrogen atom.

In some embodiments, the one or more physiochemical features of the plurality of pharmaceutical compounds include the complexity score of each of the plurality of pharmaceutical compounds. The complexity score of a compound is an estimate of how complicated the structure of the compound is, and may be based on both the elements contained in the compound and the displayed structural features of the compound including symmetry. The complexity score may be computed using the Bertz/Hendrickson/Ihlenfeldt formula.

In some embodiments, the methods provided herein comprise selecting the plurality of pharmaceutical compounds such that the complexity scores of the selected compounds define a sufficiently broad range, where the range is the difference between the highest and lowest complexity scores among the complexity scores of the selected compounds. By selecting the pharmaceutical compounds according to this criterion, the correlation between the obtained functional assay data and one or more physiochemical compound features including complexity scores may be improved. In particular, a calculated correlation may be less subject to experimental noise, may have greater statistical significance, and may be predictive of a cellular response to a wider variety of additional existing or potential pharmaceutical compounds. The plurality of pharmaceutical compounds may be selected such that the range of the complexity scores of the plurality of pharmaceutical compounds is, for example, greater than 1000, e.g., greater than 1250, greater than 1500, greater than 1750, greater than 2000, greater than 2250, greater than 2500, greater than 2750, or greater than 3000. Smaller ranges, e.g., less than 1000, are also contemplated.

In some embodiments, the one or more physiochemical features of the plurality of pharmaceutical compounds include the rotatable bond count of each of the plurality of pharmaceutical compounds. The rotatable bond count of a compound may be the sum of the single bonds of the compound, excluding single bonds that are terminal bonds, that are attached to triple bonds, or that are amide, thioamide, or sulfonamide bonds.

In some embodiments, the one or more physiochemical features of the plurality of pharmaceutical compounds include the octanol-water partition coefficient of each of the plurality of pharmaceutical compounds. The octanol-water partition coefficient, or log P, of a compound is a measure of the intrinsic lipophilicity of the compound, and may be calculated from the 2D structure of the compound using contributions from pre-defined structure fragments.

In some embodiments, the one or more physiochemical features of the plurality of pharmaceutical compounds include the aqueous solubility of each of the plurality of pharmaceutical compounds. The aqueous solubility of a compound may be a measure of the solubility of the compound in water under standard physiological conditions.

In some embodiments, the one or more physiochemical features of the plurality of pharmaceutical compounds include the pKa of each of the plurality of pharmaceutical compounds. The pKa of a compound is the pH at which an ionizable center, be it an acid or base, is present in the compound with equal proportions of the charged and uncharged forms. The pKa may be derived using the Henderson-Hasselbalch equation.

In some embodiments, the one or more physiochemical features of the plurality of pharmaceutical compounds include the plasma protein binding score of each of the plurality of pharmaceutical compounds. The plasma protein binding score of a compound may be a measure of the binding affinity of the compound to human serum albumin (HSA) and alpha-1-acidglycoprotein (AGP).

In some embodiments, the methods provided herein comprise generating one or more predicted effects on one or more cellular pathways. The one or more cellular pathways may include any known or unknown cellular pathway of interest. In some embodiments, the one or more cellular pathways include peripheral inflammation, central inflammation, mitochondrial function, second messenger signaling, generation of reactive species, or any combination thereof.

In some embodiments, generating a predicted effect of a pharmaceutical compound or a plurality of pharmaceutical compounds on a cellular pathway comprises inputting a set of functional assay data (e.g., from any of the functional assays described above) into a prediction model (e.g., a multivariate regression model or a machine-learning model) constructed for a task of predicting a cellular pathway effect as output data by applying one or more algorithms capable of modeling relationships or correlations between features of the functional assay data and the cellular pathway effect. In some embodiments, the prediction model comprises a multiple linear regression analysis. For example, the set of functional assay data may be used as predictor variables (i.e., the data from each individual functional assay be used as individual predictor variables) in a multiple linear regression analysis, wherein the dependent variable is the cellular pathway effect. In some embodiments, the prediction model comprises a multivariate regression analysis. In some embodiments, the prediction model comprises a multivariate multiple regression analysis. For example, the set of functional assay data may be used as predictor variables (i.e., the data from each individual functional assay may be used as individual predictor variables) in a multivariate regression analysis, wherein the dependent variables are effects on one or more cellular pathways.

In some embodiments, generating the predicted effect of a pharmaceutical compound or a plurality of pharmaceutical compounds on a cellular pathway comprises comparing functional assay data for the pharmaceutical compound or plurality of pharmaceutical compounds (i.e., the “experimental compound”) to functional assay data for a control pharmaceutical compound or a plurality of control pharmaceutical compounds (i.e., the “control compound”). For example, a drug with a known therapeutic outcome may be used as a control compound. In this scenario, the effect of the drug on one or more cellular pathways may already be characterized and well-understood. By comparing the functional assay data (i.e., data generated using any of the functional assays described above) for such a control compound to functional assay data for an experimental compound, one may be able to predict the effect of the experimental compound on the one or more cellular pathways. If the functional assay data for the experimental compound are similar to the functional assay data for the control compound, it would be reasonable to predict that the experimental compound would have a similar effect on one or more cellular pathways and/or a similar therapeutic outcome to the control compound.

In some embodiments of the methods described herein, the prediction models may be optimized by using functional assay data from control compounds, as described above. In some embodiments, the prediction models may be further refined by testing predictions made using the model. For example, one particular type of functional assay data (e.g., data from any of the functional assays described above) may be found to be particularly helpful in predicting the results on one specific cellular pathway (i.e., an experimental compound that has similar data to a control compound for the particular functional assay may be very likely to have a similar effect on the specific cellular pathway). In such cases, the prediction model may be weighted more heavily toward that particular type of functional assay data. As another example, one particular type of functional assay data may be found to have very little correlation to the cellular pathway effect outcome. In such cases, the prediction model may be weighted less heavily toward that particular type of functional assay data. In some embodiments, the prediction models described herein may undergo multiple rounds of optimization, either through making and testing predictions (e.g., as described above and in the Examples herein) or through inputting functional assay data for multiple control compounds.

In some embodiments, various aspects of the prediction models may be selected, optimized, or refined using one or more machine learning techniques. In some embodiments, at least one parameter of at least one of the algorithms is selected using one or more machine learning techniques. In some embodiments, the one or more machine learning techniques include a regression model, a classification model, clustering, anomaly detection, or any combination thereof. In some embodiments, any other suitable machine learning technique may be used.

In some embodiments, generating the predicted effect on a cellular pathway response comprises generating, using a prediction model (e.g., the prediction model described above), the predicted effect on the cellular pathway based on the application of the one or more algorithms described above.

In some embodiments, the methods provided herein comprise predicting, based on one or more predicted effects (e.g., predicted as described above) on one or more cellular pathways, a cellular response to a pharmaceutical compound or plurality of pharmaceutical compounds.

In some embodiments, the methods provided herein comprise predicting, based on a predicted cellular response to a pharmaceutical compound or plurality of pharmaceutical compounds, one or more therapeutic outcomes of the pharmaceutical compound or plurality of pharmaceutical compounds. In some embodiments, the methods comprise evaluating, based on the one or more predicted therapeutic outcomes, the pharmaceutical compound or plurality of pharmaceutical compounds for therapeutic application. In some embodiments, the plurality of pharmaceutical compounds comprises at least one existing medication. The therapeutic outcomes may comprise any desired outcome. In some embodiments, the one or more therapeutic outcomes include therapeutic efficacy, toxicity, side effects, interactions between the plurality of pharmaceutical compounds, or any combination thereof.

### III. Computer Program Products and Systems

In another aspect, provided herein are computer-program products tangibly embodied in non-transitory machine-readable storage media, including instructions configured to cause one or more data processors to perform specified actions. In some embodiments, the specified actions comprise obtaining a set of functional assay data for a pharmaceutical compound or a plurality of pharmaceutical compounds, wherein the functional assay data are based on one or more functional assays (e.g., any of the functional assays described above).

In some embodiments, the specified actions comprise generating one or more predicted effects on one or more cellular pathways (e.g., any of the cellular pathways described above). In some embodiments, generating a predicted effect on a cellular pathway comprises inputting a set of functional assay data into a prediction model constructed for a task of predicting a cellular pathway effect as output data by applying one or more algorithms capable of modeling relationships or correlations between features of functional assay data and the cellular pathway effect. In some embodiments, generating the predicted effect on a cellular pathway response comprises generating, using the prediction model (e.g., the prediction model described above), the predicted effect on the cellular pathway based on the application of the one or more algorithms described above.

In some embodiments, the specified actions of the computer-program products provided herein comprise predicting, based on one or more predicted effects on one or more cellular pathways, a cellular response to a pharmaceutical compound or plurality of pharmaceutical compounds.

In another aspect, provided herein is a system comprising one or more data processors and a non-transitory computer readable storage medium containing instructions which, when executed on the one or more data processors, cause the one or more data processors to perform one or more specified actions. In some embodiments, the specified actions comprise obtaining a set of functional assay data for a pharmaceutical compound or a plurality of pharmaceutical compounds, wherein the functional assay data are based on one or more functional assays (e.g., any of the functional assays described above.

In some embodiments, the specified actions comprise generating one or more predicted effects on one or more cellular pathways (e.g., any of the cellular pathways described above). In some embodiments, generating a predicted effect on a cellular pathway comprises inputting a set of functional assay data into a prediction model constructed for a task of predicting a cellular pathway effect as output data by applying one or more algorithms capable of modeling relationships or correlations between features of functional assay data and the cellular pathway effect. In some embodiments, generating the predicted effect on a cellular pathway response comprises generating, using the prediction model (e.g., the prediction model described above), the predicted effect on the cellular pathway based on the application of the one or more algorithms described above.

In some embodiments, the specified actions performed by the data processors of the systems provided herein comprise predicting, based on one or more predicted effects on one or more cellular pathways, a cellular response to a pharmaceutical compound or plurality of pharmaceutical compounds.

The present disclosure will be better understood in view of the following non-limiting examples.

### EXAMPLES

### Example 1: Functional Assays and Identification of Lead Cannabinoid Formulations

The hemp formulations outlined in Table 1 are screened. All formulations are first verified for composition and purity, using contract bioanalytical laboratory services. Concentration response curves are then performed for each formulation (concentration range (nM): 0, 1, 3, 10, 30, 100, 300, 1000). Cells are cultured at 80% confluency in 24-well plates, allowing each compound concentration to be tested in triplicate. Cultured cells are exposed to specific concentrations of compounds, and timing varies with respect to identified endpoints for subsequent description. The immune stimulating and pain toxic challenge are induced by treatment with capsaicin (CAP, 1 μM; see Kusano and Gainer, 1993, J. Neurosci. Res. 34:158-169). Controls are a standard NSAID (i.e., ibuprofen) and exposure to compounds at the various concentrations without challenge. The endpoints described below are then assessed.

TABLE 1
Cannabinoid screening formulations
represented as percent composition.

| Formulation ID | CBD | CBG | CBN | CBC |
|---|---|---|---|---|
| Sample A | 100 | — | — | — |
| Sample B | — | 100 | — | — |
| Sample C | — | — | 100 | — |
| Sample D | — | — | — | 100 |
| Sample E | 50 | 50 | — | — |
| Sample F | 50 | — | 50 | — |
| Sample G | 50 | — | — | 50 |
| Sample H | — | 50 | 50 | — |
| Sample I | — | 50 | — | 50 |
| Sample J | — | — | 50 | 50 |
| Sample K | 25 | 25 | 25 | 25 |
| Sample L | 40 | 20 | 20 | 20 |
| Sample M | 40 | 30 | 30 | — |
| Sample N | 40 | — | 30 | 30 |
| Sample O | 20 | 40 | 20 | 20 |
| Sample P | 30 | 40 | 30 | — |
| Sample Q | — | 40 | 30 | 30 |
| Sample R | 20 | 20 | 40 | 20 |
| Sample S | 30 | 30 | 40 | — |
| Sample T | — | 30 | 40 | 30 |
| Sample U | 20 | 20 | 20 | 40 |
| Sample V | 30 | 30 | — | 40 |
| Sample W | — | 30 | 30 | 40 |
| Sample X | 33 | 33 | 33 | — |
| Sample Y | 33 | — | 33 | 33 |
| Sample Z | 33 | 33 | — | 33 |
| Sample AA | — | 33 | 33 | 33 |
| Sample AB | 75 | 25 | — | — |
| Sample AC | 75 | — | 25 | — |
| Sample AD | 75 | — | — | 25 |

CBD: cannabidiol; CBG: cannabigerol; CBN: cannabinol; CBC: cannabichromene.

Cell viability: Cell viability is assessed by MTT assay (Abcam, ab211091). Two plates are prepared for each compound. Plated cells are exposed to compounds and controls, in triplicate, following the concentration curve outlined above. Two hours following compound treatment, one plate is treated with CAP, and the other plate serves as a no-CAP control. MTT assays are conducted at 24 hours to determine impact on cell proliferation and viability.

Cytokine production: Supernatant from each well of the cell viability assay above is collected and cytokine production is assessed. Cytokine-specific ELISAs is used to measure IL-1β, TNFα, and IL-6 (all kits available at Abcam). mRNA for these and additional cytokines is also quantified using real-time PCR analysis.

Mitochondrial function: The Seahorse XFe96 Metabolic Flux analyzer, capable of real-time analysis of mitochondrial respiration, is used to assess mitochondrial function using various preparations (see Niesman et al., 2013, Mol. Cell Neurosci. 56:283-297). This system has 96 wells, six of which serve as internal plate controls, leaving 90 effective assay wells. Each well has 4 injector ports that can deliver various reagents (i.e., serum, chemicals, etc.) at specified times in the assay cycle, and specific assays probe mitochondrial stress. Timing of treatment is as above.

Generation of reactive oxygen/nitrogen species (RONS): RONS are assessed by live-cell plate reader assays and electron paramagnetic resonance (EPR). As EPR is labor-intensive, the plate reader-based assay is used initially to measure mitochondrial superoxide (MitoSOX assay, Thermo Fisher, M36008) and nitric oxide (Cell Biolabs, OxiSelect NO Assay, STA-800), as both reactive species have been linked to pain and immune cell function. Once particular concentrations and compounds have been identified, a more detailed and sophisticated EPR approach identifies hydroxyl, superoxide, and nitric oxide species, using selective spin probes (see Fridolfsson et al., 2012, FASEB J. 26:4637-4649 and Hogg, 2010, Free Radic. Biol. Med. 49:122-129). As RONS are short-lived, this assay involves pre-incubation of cells with compounds for 2 hours, followed by CAP induction of stress. Such reactive species are assessed 10 min post-CAP treatment.

Cyclic-AMP synthesis: Cyclic-AMP (cAMP) is a classic second messenger system utilized by cells to initiate signaling cascades. cAMP is assessed by radioimmunoassay (see Yokoyama et al., 2008, Proc. Nall. Acad. Sci. USA 105:6386-6391) As second messenger systems are short-lived, this assay involves pre-incubation of cells with compounds for 2 hours, followed by CAP induction of stress. cAMP is assessed 10 min post-CAP treatment.

Lead compounds (up to 5) are validated on primary cells isolated from rats (dorsal root ganglion, Schwann cells, and microglia) for the 5 assays proposed above. A platform is developed to integrate, create a matrix of the endpoints, and ultimately analyze the correlated data sets to identify two optimal formulations for advancement to further testing. As data are generated, they are analyzed. As all in vitro data sets are available, multivariate comparisons are performed to account for changes in the multiple assays. Compounds with changes in multiple parameters undergo a more in-depth analysis of the efficacy at specific concentrations to find those combinations that may have optimal effects for in vivo testing. Compounds with favorable concentration profiles that impact at least 3 out of 5 parameters are tested further, identifying those specific formulations that impact multiple endpoints in a concentration-specific manner. As these endpoints result from interconnected mechanisms, compounds that impact one mechanism may impact another. Various cannabinoids that impact different endpoints and combination formulations that have a more robust overall impact in terms of efficacy are identified.

### Example 2: Screening of FDA-Approved Compounds to Build Prediction Platform for Unknown Compounds

A platform is developed that is referenced and informed by known compounds with clear mechanisms of action and disease modulating properties. The APExBIO DiscoveryProbe FDA-Approved Library (L1021) that contains 1971 FDA approved compounds is utilized. The reference assays run these compounds on the existing assays described above using various cells representing key organs (brain, heart, lung, muscle, kidney, and liver). This serves as a repository for comparison of unknown compounds that are screened. The screening enables suggestions for disease targets and some efficacy measures based on assays performed to predict optimal candidates for drug discovery advancement.

### Example 3: Calculating and Evaluating Correlations Between Cell Metabolic Responses and Physiochemical Features of Pharmaceutical Compounds

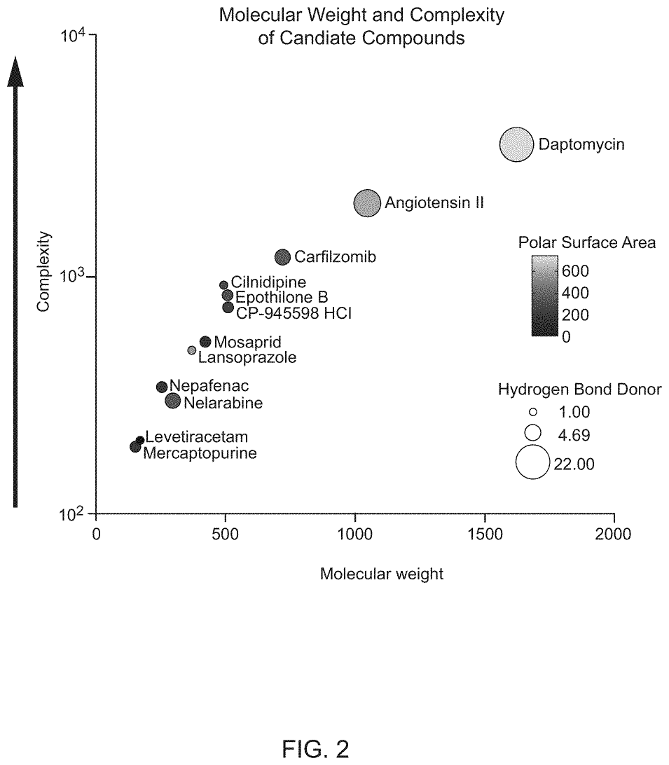

A panel of twelve pharmaceutical compounds is selected, such that the compounds vary in their physiochemical features of complexity, polarity, and size (FIG. 2). As shown in FIG. 2, the complexity scores represented by the twelve selected compounds range from approximately 2×10² to 5×10³. The molecular weights of the twelve compounds range from approximately 200 g/mol to approximately 1700 g/mol. The polar surface areas of the compounds range from approximately 0 to approximately 600. The hydrogen bond donor counts of the compounds range from approximately 1 to approximately 22.

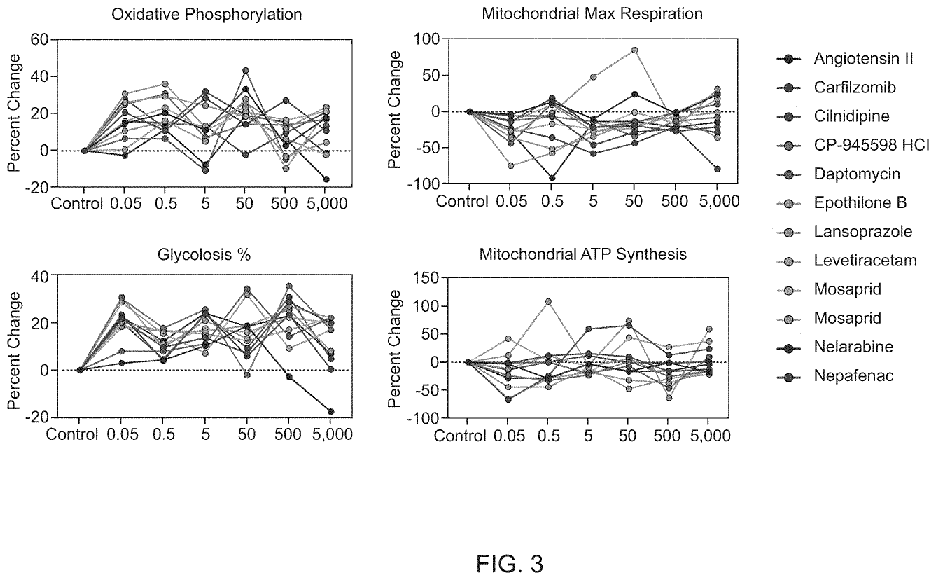

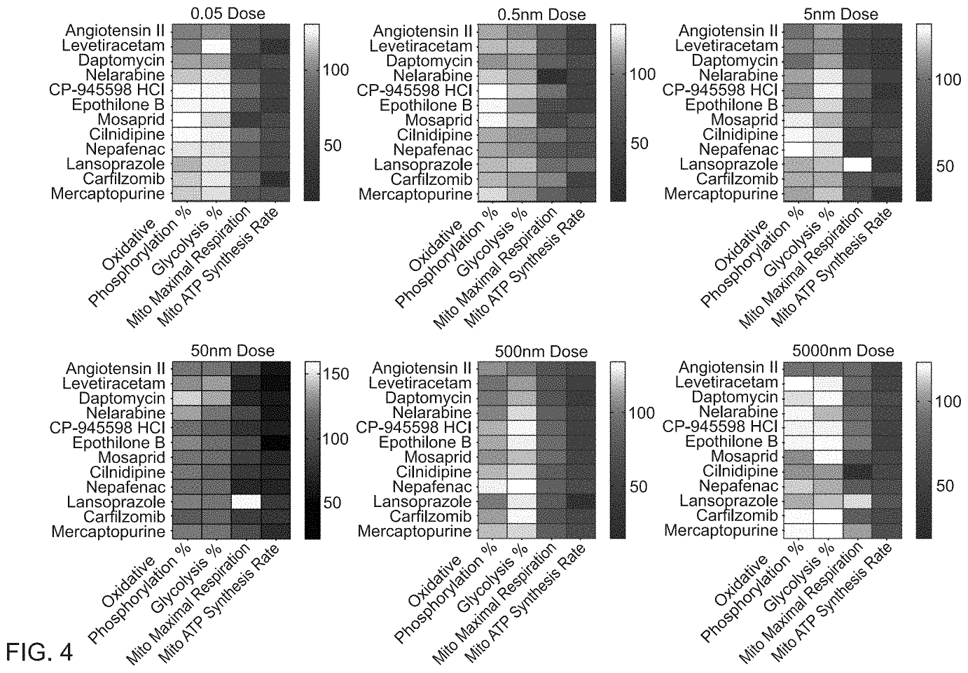

Different doses ranging from 0.05 nM to 5000 nM of the twelve selected compounds are delivered to populations of cells, and oxidative phosphorylation, glycolysis, mitochondrial respiration, and mitochondrial ATP synthesis assays are used to measure responses of the cell populations to the different compound types and concentrations (FIGS. 3 and 4). The graphs of FIG. 3 and FIG. 4 plot the percent differences of these responses in relation to a control response for each assay, compound, and dose. The results show that a dose of 0.05 nm can be selected to provide a significant variance among responses, while also reducing or eliminating a risk of cellular toxicity associated with higher doses of pharmaceutical compounds.

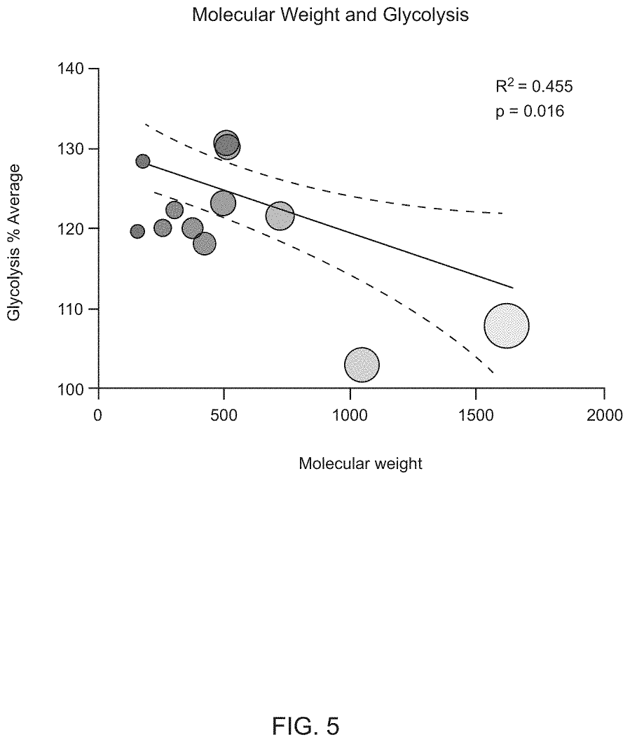

Correlations are calculated relating the oxidative phosphorylation, glycolysis, mitochondrial respiration, and mitochondrial ATP synthesis assay data to different combinations of the physiochemical features of the twelve compounds. For example, FIG. 5 plots the correlation between the cellular functional response of glycolysis and the molecular weight of the compounds. The resulting calculated P-value of 0.016 for the correlation demonstrates the statistical significance of correlations that can be identified using the methods and systems provided herein.

### ILLUSTRATIONS

Illustration 1: A method comprising: obtaining a set of functional assay data for a pharmaceutical compound or a plurality of pharmaceutical compounds, wherein the functional assay data are based on one or more functional assays; generating one or more predicted effects on one or more cellular pathways, wherein generating a predicted effect on a cellular pathway comprises: inputting the set of functional assay data into a prediction model constructed for a task of predicting a cellular pathway effect as output data by applying one or more algorithms capable of modeling relationships or correlations between features of functional assay data and the cellular pathway effect; generating, using the prediction model, the predicted effect on the cellular pathway based on the application of the one or more algorithms; predicting, based on the one or more predicted effects on the one or more cellular pathways, a cellular response to the pharmaceutical compound or plurality of pharmaceutical compounds.

Illustration 2: The method of illustration 1, wherein the prediction model comprises a regression analysis.

Illustration 3: The method of illustration 2, wherein the regression analysis is a linear regression analysis, a multiple linear regression analysis, a multivariate regression analysis, a multivariate multiple regression analysis, or a combination thereof.

Illustration 4: The method of any one of illustrations 1 to 3, wherein the one or more algorithms are selected using one or more machine learning techniques.

Illustration 5: The method of any one of illustrations 1 to 4, wherein at least one parameter of at least one algorithm is selected using one or more machine learning techniques.

Illustration 6: The method of illustration 4 or 5, wherein the one or more machine learning techniques include: a) a regression model; b) a classification model; c) clustering; or d) anomaly detection.

Illustration 7: The method of any one of illustrations 1 to 6, wherein i) the pharmaceutical compound is an existing compound; or ii) the plurality of pharmaceutical compounds comprises existing compounds.

Illustration 8: The method of any one of illustrations 1 to 7, wherein the one or more functional assays are performed on cells in culture.

Illustration 9: The method of any one of illustrations 1 to 8, wherein the one or more functional assays include: a) a cell viability assay; b) a cytokine production assay; c) a mitochondrial function assay; d) a reactive oxygen/nitrogen species generation assay; e) a cyclic-AMP synthesis assay; f) a gene expression assay; g) a cell immune response assay; or h) combinations thereof.

Illustration 10: The method of illustration 9, wherein the one or more functional assays include at least two of: a) a cell viability assay; b) a cytokine production assay; c) a mitochondrial function assay; d) a reactive oxygen/nitrogen species generation assay; e) a cyclic-AMP synthesis assay; f) a gene expression assay; g) a cell immune response assay; or h) combinations thereof.

Illustration 11: The method of illustration 9, wherein the one or more functional assays include at least three of: a) a cell viability assay; b) a cytokine production assay; c) a mitochondrial function assay; d) a reactive oxygen/nitrogen species generation assay; e) a cyclic-AMP synthesis assay; f) a gene expression assay; g) a cell immune response assay; or h) combinations thereof.

Illustration 12: The method of illustration 9, wherein the one or more functional assays include: a) a cell viability assay; b) a cytokine production assay; c) a mitochondrial function assay; d) a reactive oxygen/nitrogen species generation assay; e) a cyclic-AMP synthesis assay; f) a gene expression assay; and g) a cell immune response assay.

Illustration 13: The method of any one of illustrations 1 to 8, wherein the one or more functional assays include: a) a cell viability assay; b) a cytokine production assay; c) a mitochondrial function assay; d) a reactive oxygen/nitrogen species generation assay; e) a cyclic-AMP synthesis assay; or f) combinations thereof.

Illustration 14: The method of illustration 13, wherein the one or more functional assays include at least two of: a) a cell viability assay; b) a cytokine production assay; c) a mitochondrial function assay; d) a reactive oxygen/nitrogen species generation assay; e) a cyclic-AMP synthesis assay; or f) combinations thereof.

Illustration 15: The method of illustration 13, wherein the one or more functional assays include at least three of: a) a cell viability assay; b) a cytokine production assay; c) a mitochondrial function assay; d) a reactive oxygen/nitrogen species generation assay; e) a cyclic-AMP synthesis assay; or f) combinations thereof.

Illustration 16: The method of illustration 13, wherein the one or more functional assays include at least three of: a) a cell viability assay; b) a cytokine production assay; c) a mitochondrial function assay; d) a reactive oxygen/nitrogen species generation assay; and e) a cyclic-AMP synthesis assay.

Illustration 17: The method of any one of illustrations 1 to 16, wherein the one or more cellular pathways include: a) peripheral inflammation; b) central inflammation; c) mitochondrial function; d) second messenger signaling; e) generation of reactive species; or f) combinations thereof.

Illustration 18: The method of any one of illustrations 1 to 17, further comprising: predicting, based on the predicted cellular response to the pharmaceutical compound or plurality of pharmaceutical compounds, one or more therapeutic outcomes of the pharmaceutical compound or plurality of pharmaceutical compounds; evaluating, based on the one or more predicted therapeutic outcomes, the pharmaceutical compound or plurality of pharmaceutical compounds for therapeutic application.

Illustration 19: The method of illustration 18, wherein the plurality of pharmaceutical compounds comprises at least one existing medication.

Illustration 20: The method of illustration 18 or 19, wherein the one or more therapeutic outcomes include: a) therapeutic efficacy; b) toxicity; c) side effects; d) interactions between the plurality of pharmaceutical compounds, or e) combinations thereof.

Illustration 21: A computer-program product tangibly embodied in a non-transitory machine-readable storage medium, including instructions configured to cause one or more data processors to perform actions including: obtaining a set of functional assay data for a pharmaceutical compound or a plurality of pharmaceutical compounds, wherein the functional assay data are based on one or more functional assays; generating one or more predicted effects on one or more cellular pathways, wherein generating a predicted effect on a cellular pathway comprises: inputting the set of functional assay data into a prediction model constructed for a task of predicting a cellular pathway effect as output data by applying one or more algorithms capable of modeling relationships or correlations between features of functional assay data and the cellular pathway effect; generating, using the prediction model, the predicted effect on the cellular pathway based on the application of the one or more algorithms; predicting, based on the one or more predicted effects on the one or more cellular pathways, a cellular response to the pharmaceutical compound or plurality of pharmaceutical compounds.

Illustration 22: A system comprising: one or more data processors; and a non-transitory computer readable storage medium containing instructions which, when executed on the one or more data processors, cause the one or more data processors to perform actions including: obtaining a set of functional assay data for a pharmaceutical compound or a plurality of pharmaceutical compounds, wherein the functional assay data are based on one or more functional assays; generating one or more predicted effects on one or more cellular pathways, wherein generating a predicted effect on a cellular pathway comprises: inputting the set of functional assay data into a prediction model constructed for a task of predicting a cellular pathway effect as output data by applying one or more algorithms capable of modeling relationships or correlations between features of functional assay data and the cellular pathway effect; generating, using the prediction model, the predicted effect on the cellular pathway based on the application of the one or more algorithms; predicting, based on the one or more predicted effects on the one or more cellular pathways, a cellular response to the pharmaceutical compound or plurality of pharmaceutical compounds.

Illustration 23: A method comprising: obtaining a set of functional assay data for a plurality of pharmaceutical compounds, wherein the functional assay data are based on one or more functional assays performed on each of a plurality of populations of cells, wherein each of the plurality of populations of cells has independently been contacted with a dose of one of the plurality of pharmaceutical compounds; calculating, for each of the one or more functional assays, a parameter describing a correlation between (1) one or more physiochemical features of the plurality of pharmaceutical compounds, and (2) the functional assay data for the functional assay; and determining which of the calculated parameters satisfy a significance threshold.

Illustration 24: The method of illustration 23, wherein the calculating comprises performing a regression analysis.

Illustration 25: The method of illustration 24, wherein the regression analysis is a linear regression analysis, a multiple linear regression analysis, a multivariate regression analysis, a multivariate multiple regression analysis, or a combination thereof.

Illustration 26: The method of illustration 24 or 25, wherein the parameter comprises a P-value.

Illustration 27: The method of illustration 26, wherein the determining comprises evaluating which of the calculated P-values are less than 0.05.

Illustration 28: The method of any one of illustrations 23 to 27, wherein the plurality of pharmaceutical compounds comprises existing compounds.

Illustration 29: The method of any one of illustrations 23 to 28, wherein at least one of the plurality of pharmaceutical compounds is a non-naturally-occurring compound.

Illustration 30: The method of any one of illustrations 23 to 29, wherein the one or more physiochemical features comprise: the molecular weight of each of the plurality of pharmaceutical compounds; the polar surface area of each of the plurality of pharmaceutical compounds; the hydrogen bond donor count of each of the plurality of pharmaceutical compounds; the hydrogen bond acceptor count of each of the plurality of pharmaceutical compounds; the complexity score of each of the plurality of pharmaceutical compounds; the rotatable bond count of each of the plurality of pharmaceutical compounds; the octanol-water partition coefficient of each of the plurality of pharmaceutical compounds; the aqueous solubility of each of the plurality of pharmaceutical compounds; the pKa value of each of the plurality of pharmaceutical compounds; the plasma protein binding score of each of the plurality of pharmaceutical compounds; or a combination thereof.

Illustration 31: The method of illustration 30, wherein the one or more physiochemical features comprise at least two of: the molecular weight of each of the plurality of pharmaceutical compounds; the polar surface area of each of the plurality of pharmaceutical compounds; the hydrogen bond donor count of each of the plurality of pharmaceutical compounds; the hydrogen bond acceptor count of each of the plurality of pharmaceutical compounds; the complexity score of each of the plurality of pharmaceutical compounds; the rotatable bond count of each of the plurality of pharmaceutical compounds; the octanol-water partition coefficient of each of the plurality of pharmaceutical compounds; the aqueous solubility of each of the plurality of pharmaceutical compounds; the pKa value of each of the plurality of pharmaceutical compounds; and the plasma protein binding score of each of the plurality of pharmaceutical compounds.

Illustration 32: The method of illustration 30, wherein the one or more physiochemical features comprise at least three of: the molecular weight of each of the plurality of pharmaceutical compounds; the polar surface area of each of the plurality of pharmaceutical compounds; the hydrogen bond donor count of each of the plurality of pharmaceutical compounds; the hydrogen bond acceptor count of each of the plurality of pharmaceutical compounds; the complexity score of each of the plurality of pharmaceutical compounds; the rotatable bond count of each of the plurality of pharmaceutical compounds; the octanol-water partition coefficient of each of the plurality of pharmaceutical compounds; the aqueous solubility of each of the plurality of pharmaceutical compounds; the pKa value of each of the plurality of pharmaceutical compounds; and the plasma protein binding score of each of the plurality of pharmaceutical compounds.

Illustration 33: The method of illustration 30, wherein the one or more physiochemical features comprise the molecular weight of each of the plurality of pharmaceutical compounds.

Illustration 34: The method of any one of illustrations 23 to 33, wherein the one or more functional assays comprise: a) a cell viability assay; b) a cytokine production assay; c) a mitochondrial function assay; d) a reactive oxygen/nitrogen species generation assay; e) a cyclic-AMP synthesis assay; f) a gene expression assay; g) a cell immune response assay; or h) a combination thereof.

Illustration 35: The method of illustration 34, wherein the one or more functional assays comprise at least two of: a) a cell viability assay; b) a cytokine production assay; c) a mitochondrial function assay; d) a reactive oxygen/nitrogen species generation assay; e) a cyclic-AMP synthesis assay; f) a gene expression assay; and g) a cell immune response assay.

Illustration 36: The method of illustration 34, wherein the one or more functional assays comprise at least three of: a) a cell viability assay; b) a cytokine production assay; c) a mitochondrial function assay; d) a reactive oxygen/nitrogen species generation assay; e) a cyclic-AMP synthesis assay; f) a gene expression assay; and g) a cell immune response assay.

Illustration 37: The method of illustration 34, wherein the one or more functional assays comprise: a) a cell viability assay; b) a cytokine production assay; c) a mitochondrial function assay; d) a reactive oxygen/nitrogen species generation assay; e) a cyclic-AMP synthesis assay; f) a gene expression assay; and g) a cell immune response assay.

Illustration 38: The method of any one of illustrations 23 to 34, wherein the one or more functional assays comprise: a) a cell viability assay; b) a cytokine production assay; c) a mitochondrial function assay; d) a reactive oxygen/nitrogen species generation assay; e) a cyclic-AMP synthesis assay; or f) combinations thereof.

Illustration 39: The method of illustration 38, wherein the one or more functional assays comprise at least two of: a) a cell viability assay; b) a cytokine production assay; c) a mitochondrial function assay; d) a reactive oxygen/nitrogen species generation assay; and e) a cyclic-AMP synthesis assay.

Illustration 40: The method of illustration 38, wherein the one or more functional assays comprise at least three of: a) a cell viability assay; b) a cytokine production assay; c) a mitochondrial function assay; d) a reactive oxygen/nitrogen species generation assay; and e) a cyclic-AMP synthesis assay.

Illustration 41: The method of any one of illustrations 23 to 34, wherein the one or more functional assays comprise a mitochondrial function assay.

Illustration 42: The method of any one of illustrations 23 to 34, wherein the one or more functional assays comprise a glycolysis assay.

Illustration 43: The method of any one of illustrations 23-42, further comprising: selecting the plurality of pharmaceutical compounds, such that the range of the molecular weights of the plurality of pharmaceutical compounds is greater than 500 g/mol.

Illustration 44: The method of any one of illustrations 23-43, further comprising: selecting the plurality of pharmaceutical compounds, such that the range of the hydrogen bond donor counts of the plurality of pharmaceutical compounds is greater than 5.

Illustration 45: The method of any one of illustrations 23-44, further comprising: selecting the plurality of pharmaceutical compounds, such that the range of the polar surface areas of the plurality of pharmaceutical compounds is greater than 200 square angstroms per molecule.

Illustration 46: The method of any one of illustrations 23-45, further comprising: selecting the plurality of pharmaceutical compounds, such that the range of the complexity scores of the plurality of pharmaceutical compounds is greater than 1000.

Illustration 47: The method of any one of illustrations 23-46, further comprising: selecting the dose, such that, for each of the one or more functional assays, the absolute range of the percent differences from control for the functional assay data is greater than 10%.

Illustration 48: A computer-program product tangibly embodied in a non-transitory machine-readable storage medium, including instructions configured to cause one or more data processors to perform actions including: obtaining a set of functional assay data for a plurality of pharmaceutical compounds, wherein the functional assay data are based on one or more functional assays performed on each of a plurality of populations of cells, wherein each of the plurality of populations of cells has independently been contacted with a dose of one of the plurality of pharmaceutical compounds; calculating, for each of the one or more functional assays, a parameter describing a correlation between (1) one or more physiochemical features of the plurality of pharmaceutical compounds, and (2) the functional assay data for the functional assay; and determining which of the calculated parameters satisfy a significance threshold.

Illustration 49: A system comprising: one or more data processors; and a non-transitory computer readable storage medium containing instructions which, when executed on the one or more data processors, cause the one or more data processors to perform actions including: obtaining a set of functional assay data for a plurality of pharmaceutical compounds, wherein the functional assay data are based on one or more functional assays performed on each of a plurality of populations of cells, wherein each of the plurality of populations of cells has independently been contacted with a dose of one of the plurality of pharmaceutical compounds; calculating, for each of the one or more functional assays, a parameter describing a correlation between (1) one or more physiochemical features of the plurality of pharmaceutical compounds, and (2) the functional assay data for the functional assay; and determining which of the calculated parameters satisfy a significance threshold.

While the disclosure has been described in detail, modifications within the spirit and scope of the disclosure will be readily apparent to those of skill in the art. It should be understood that aspects of the disclosure and portions of various embodiments and various features recited above and/or in the appended claims may be combined or interchanged either in whole or in part. In the foregoing descriptions of the various embodiments, those embodiments which refer to another embodiment may be appropriately combined with other embodiments as will be appreciated by one of ordinary skill in the art. Furthermore, those of ordinary skill in the art will appreciate that the foregoing description is by way of example only, and is not intended to limit the disclosure.

1. A method comprising:
obtaining a set of functional assay data for a plurality of pharmaceutical compounds, wherein the functional assay data are based on one or more functional assays performed on each of a plurality of populations of cells, wherein each of the plurality of populations of cells has independently been contacted with a dose of one of the plurality of pharmaceutical compounds;
calculating, for each of the one or more functional assays, a parameter describing a correlation between (1) one or more physiochemical features of the plurality of pharmaceutical compounds, and (2) the functional assay data for the functional assay; and
determining which of the calculated parameters satisfy a significance threshold.

2. The method of claim 1, wherein the calculating comprises performing a regression analysis.

3. The method of claim 2, wherein the regression analysis is a linear regression analysis, a multiple linear regression analysis, a multivariate regression analysis, a multivariate multiple regression analysis, or a combination thereof.

4. The method of claim 2, wherein the parameter comprises a P-value.

5. The method of claim 4, wherein the determining comprises evaluating which of the calculated P-values are less than 0.05.

6. The method of claim 1, wherein the plurality of pharmaceutical compounds comprises existing compounds.

7. The method of claim 1, wherein at least one of the plurality of pharmaceutical compounds is a non-naturally-occurring compound.

8. The method of claim 1, wherein the one or more physiochemical features comprise:
a) the molecular weight of each of the plurality of pharmaceutical compounds;
b) the polar surface area of each of the plurality of pharmaceutical compounds;
c) the hydrogen bond donor count of each of the plurality of pharmaceutical compounds;
d) the hydrogen bond acceptor count of each of the plurality of pharmaceutical compounds;
e) the complexity score of each of the plurality of pharmaceutical compounds;
f) the rotatable bond count of each of the plurality of pharmaceutical compounds;
g) the octanol-water partition coefficient of each of the plurality of pharmaceutical compounds;
h) the aqueous solubility of each of the plurality of pharmaceutical compounds;
i) the pKa value of each of the plurality of pharmaceutical compounds;
j) the plasma protein binding score of each of the plurality of pharmaceutical compounds; or
k) a combination thereof.

9. The method of claim 8, wherein the one or more physiochemical features comprise the molecular weight of each of the plurality of pharmaceutical compounds.

10. The method of claim 1, wherein the one or more functional assays comprise:
a) a cell viability assay;
b) a cytokine production assay;
c) a mitochondrial function assay;
d) a reactive oxygen/nitrogen species generation assay;
e) a cyclic-AMP synthesis assay;
f) a gene expression assay;
g) a cell immune response assay; or
combinations thereof.

11. The method of claim 1, wherein the one or more functional assays comprise a mitochondrial function assay.

12. The method of claim 1, wherein the one or more functional assays comprise a glycolysis assay.

13. The method of claim 2, further comprising:
selecting the plurality of pharmaceutical compounds, such that the range of the molecular weights of the plurality of pharmaceutical compounds is greater than 500 g/mol.

14. The method of claim 1, further comprising:
selecting the plurality of pharmaceutical compounds, such that the range of the hydrogen bond donor counts of the plurality of pharmaceutical compounds is greater than 5.

15. The method of claim 1, further comprising:
selecting the plurality of pharmaceutical compounds, such that the range of the polar surface areas of the plurality of pharmaceutical compounds is greater than 200 square angstroms per molecule.

16. The method claim 1, further comprising:
selecting the plurality of pharmaceutical compounds, such that the range of the complexity scores of the plurality of pharmaceutical compounds is greater than 1000.

17. The method of claim 1, further comprising:
selecting the dose, such that, for each of the one or more functional assays, the absolute range of the percent differences from control for the functional assay data is greater than 10%.

18. A computer-program product tangibly embodied in a non-transitory machine-readable storage medium, including instructions configured to cause one or more data processors to perform actions including:
obtaining a set of functional assay data for a plurality of pharmaceutical compounds, wherein the functional assay data are based on one or more functional assays performed on each of a plurality of populations of cells, wherein each of the plurality of populations of cells has independently been contacted with a dose of one of the plurality of pharmaceutical compounds;
calculating, for each of the one or more functional assays, a parameter describing a correlation between (1) one or more physiochemical features of the plurality of pharmaceutical compounds, and (2) the functional assay data for the functional assay; and
determining which of the calculated parameters satisfy a significance threshold.

19. A system comprising:
one or more data processors; and
a non-transitory computer readable storage medium containing instructions which, when executed on the one or more data processors, cause the one or more data processors to perform actions including:
obtaining a set of functional assay data for a plurality of pharmaceutical compounds, wherein the functional assay data are based on one or more functional assays performed on each of a plurality of populations of cells, wherein each of the plurality of populations of cells has independently been contacted with a dose of one of the plurality of pharmaceutical compounds;
calculating, for each of the one or more functional assays, a parameter describing a correlation between (1) one or more physiochemical features of the plurality of pharmaceutical compounds, and (2) the functional assay data for the functional assay; and
determining which of the calculated parameters satisfy a significance threshold.
